(19) United States
(12) Patent Application Publication
(10) Pub. No.: US 2008/0019904 A1
(43) Pub. Date: Jan. 24, 2008

(54) System For Manufacturing Micro-Sheres

(75) Inventors: Marcel Rene Boehmer, Eindhoven (NL); Hendrik Roelof Stapert, Eindhoven (NL); Paulus Cornelis Duineveld, Drachten (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/570,787

(22) PCT Filed: Jun. 24, 2005
(86) PCT No.: PCT/IB05/52098

§ 371 Date: Dec. 18, 2006

(30) Foreign Application Priority Data
Jun. 29, 2004 (EP) 04103038.8

(51) Int. Cl. A61K 49/22; A61K 49/18; A61K 51/12; A61K 9/50; B01J 13/04

(52) U.S. Cl. 424/1.29; 264/4.3; 424/490; 424/9.37; 424/9.5

(57) ABSTRACT

System for manufacturing micro-spheres of a production fluid (23) containing a constituting material. The system comprises a reservoir (1) for holding a receiving fluid (11). There further is provided a jetting module (2) having at least one nozzle (21) for jetting the production fluid into the receiving fluid. The production fluid contains a concentration of the constituting material in the range between] and 0.01 and 5%. The constituent(s) of the final microspheres are dissolved in the production fluid. As a nozzle an ink-jet head is employed that is placed under the surface of the receiving liquid/air interface. In this configuration inkjetted droplets do not have to pass the air-liquid interface but will be injected directly into the receiving fluid.

The invention pertains to a system for manufacturing micro-spheres from a production fluid.

Such a system is known from the paper ‘Uniform Paclitaxel-loaded biodegradable microspheres manufactured by ink-jet technology’ in Proc. Recent Adv. in Drug Delivery Sys. (March 2003) by D. Radulescu et al.

The known system produces biodegradable microspheres, i.e. micro-spheres on the basis of ink-jet technology. In particular paclitaxel loaded PLGA microspheres of narrow size distribution and controlled diameter are manufactured. The known system employs a drop-on-demand process or pressure assisted drop-on-demand for jetting a paclixatel PLGA mixture into an aqueous polyvinyl alcohol solution. Microspheres having a narrow size distribution around 60 μm±1 μm have been produced. These micro-spheres are formed from a dichloroethane solution containing 3% of PLGA and 1.5% of paclitaxel. After making drops of this solution the dichloroethane is removed and solid particles containing a mixture of PLGA and paclitaxel remain.

An object of the invention is to provide a system to manufacturing micro-spheres having far smaller sizes than the size of the microspheres produced by the known system and also achieving narrow size distribution.

The invention is based on the insight that starting from low concentration, i.e. in the range of 0.01% to 5%, from polymers monodisperse, dense polymer particles can be formed by inkjetting and subsequent removal of solvent. Good results are achieved in the range of polymer concentration of 0.01 to 3%. Particularly reliable formation of monodisperse microspheres is achieved in the range of polymer concentration of 0.01 to 2.9%. The size of the micro-spheres bubbles is very small, notably micro-spheres having size in the range 1-15 μm, with a small variation in volume of about 3% is achieved. Typically 5 μm sized micro-spheres are produced.

The production fluid is a solution of the constituting material, i.e. the material(s) of which the microspheres are to be made in a solvent. In other words: the constituent(s) of the final microspheres are dissolved in the production fluid. For example in the solvent polymer or monomers may be dissolved. The solvent in the production fluid should have a limited solubility in the receiving fluid with the receiving fluid. The solvent will slowly diffuse into the receiving fluid and subsequently evaporate, leading to shrinkage of the drops of the production fluid. Good results are achieved at solubilities around 1%, such as is the case for dichloroethane (DCE) or dichloroomethane (DCM) in water.

Good maintenance of the size and distribution of the size of the micro-spheres is in particular achieved when the micro-spheres form a stable colloid, which is facilitated by the presence of polymers or surfactant in the receiving fluid. Then coalescence of droplets into larger droplets is counteracted/prevented. In a preferred embodiment the production liquid contains a halogenated solvent which has a high density, such as dichloro-ethane and the receiving solution is aqueous. Halogenated solvents with a small solubility in water (about 0.8% for dichloroethane) and a high vapour pressure are preferred for slow and controlled removal from the drops of production fluid. The constituents of the final microspheres are dissolved in the production fluid. For constituents to be used (intravenously) inside living humans, biodegradable polymers and (modified) phospholipids are preferred as carrier materials, drugs and imaging agents can be incorporated in the microspheres and targeted to markers of diseases expressed on blood vessel walls, such as markers for angiogenesis associated with tumours and markers for vulnerable plaques. After jetting, the excess stabilizer can be removed through a series of washing steps and the removal of the final remainders of the halogenated solvent can be established by lyophilization (freeze drying).

It appears an essentially monodisperse distribution of small sized microspheres is achieved. The jetting of the production fluid into the receiving fluid leads to better excellent separation of the individual micro-droplets when they leave the nozzle. The manufacturing involves jetting of the production fluid at relatively high jetting rates, into a receiving fluid. It is found that at low polymer concentration in the production fluid, shrinkage of the droplet to essentially non-porous polymer micro-spheres occurs.

As the method outlined above leads to dense particles, it will also lead to dense shells, therefore giving a robust encapsulation of liquids or gases. To achieve this the production liquid has to be modified with a non-solvent for the shell forming material. The production liquid can also be modified to include phospholipids rather than polymers or a combination of phospholipids and polymers.

According to another aspect of the invention the system for manufacturing micro-spheres is provided with a control system to operate the jetting in a pulsed fashion. The control system control the application of excitation pulses to the jetting module. Block shaped pulses achieve good results in that somewhat larger sized micro-spheres of a few tenths of nl volume are produced.

According to a further aspect of the invention, the jetting system is provided with several nozzles that can be individually controlled to adjust the sizes of the micro-bubbles from the respective nozzles. For example, these nozzles are controlled so that they all produce bubbles within a narrow size distribution. The individual control of the individual nozzles then compensates for small differences between the nozzles. Notably, this is achieved by adjusting the electrical activation pulses applied to the nozzles. In particular, the width of the volume distribution can be narrowed to about 3-5%. As more nozzles are employed, more micro-spheres can be produced per unit of time.

According to another aspect of the invention, micro-spheres with a controlled porosity can be formed. According to a further aspect of the invention, the reservoir is provided with a temperature control to cool the receiving fluid below its condensation temperature. Good results are achieved when the receiving liquid is cooled below room temperature, i.e. below 298K. Then, the production fluid is jetted in the form of droplets into the cooled receiving liquid, and may be stored for later use. When the temperature of the droplets is raised, the receiving fluid is evaporated and gas-filled micro-spheres are formed. Further a catalyst may be employed in the receiving liquid to initiate polymerization of the production fluid to enhance formation of stable micro-bubbles. As an alternative irradiation with electromagnetic radiation, for example ultra-violet radiation of the bubbles leaving the nozzle by means of an irradiation module may be employed for photo-initiation of polymerization.

In another aspect of the invention one can make use of the lower critical solution temperature (LCST) or upper critical solution temperature (UCST) of polymers. An LCST is observed when precipitation of the polymer occurs at increasing temperatures. Thus, for production of micro-spheres, the temperature of the receiving fluid is raised above the LCST and the polymer containing solution is jetted at temperature below the LCST. Micro-spheres will then form due to the precipitation of the polymer within the well-defined droplets. This approach is particular advantageous when use of halogenated receiving liquids is not allowed, or when lyophilization (freeze-drying) is not desired. Example of a well-known polymer with an LCST is poly(N-isopropylacryl amide)(PNiPAAm). The LCST of this polymer (˜32° C.) can be easily tuned to relevant temperatures for clinical application (e.g. below or above 37° C.) by copolymerisation with poly(acrylic acid) or more hydrophobic acrylates, depending on the LCST desired.

When the droplets are jetted into air, instead of directly into the receiving liquid, then employing a long flight path—e.g. of a few centimeters—from the nozzle for the droplets also leads to formation of micro-spheres.

According to one aspect of the invention the ink-jet head is placed under the surface of the receiving liquid/air interface. In this configuration inkjetted droplets do not have to pass the air-liquid interface but will be injected directly into the receiving fluid. Using this configuration the stabilizing action of polymers or surfactants present in the receiving liquid will be optimized leading to a stable emulsion of drops of the production fluid in the receiving liquid. Alternatively, the stabilizer can be added to the production fluid, a suitable stabilizer is a phospholipid. As an additional advantage of submerged inkjetting no problems associated with the surface characteristics of the receiving liquid will occur. Good emulsion and jetting stability are supported by the production fluid and the receiving liquid having different densities. If the production fluid has a higher density than the receiving liquid and the jet is in the direction of gravity, the droplet will continue to sink to the bottom of the container with their sedimentation velocity, from which they can be easily collected. In an alternative set-up the production fluid has a lower density than the receiving liquid and the droplets are jetted in a direction such that the droplets float towards the surface of the receiving liquid without returning towards the nozzle. The micro-spheres that are formed can then be collected at the surface of the receiving liquid.

The invention also relates to an ultra-sound contrast agent. The use of apsherical microdroplets as an ultra-sound contrast agent is known per se from the U.S. Pat. No. 5,606,973. The ultra-sound contrast of the invention comprises essentially mono-disperse micro-bubbles filled with a gas or monodisperse microspheres filled with fluorocarbonliquid. The micro-bubbles not only change the reflection of ultra sound, but also are able to resonate in the ultrasound field which yields harmonics. Such a mono-disperse contrast agent is in particular advantageous to be employed in the form of a targeted contrast agent. The targeted contrast agent selectively binds to specific receptors, e.g. adheres to vessel wall tissue. The resonance frequency of selectively bound micro-bubbles is shifted with respect to the non-bound micro-bubbles. The mono-disperse distribution of micro-bubbles leads to narrow line width of these resonances and hence the frequency shift can be detected. Hence, bound contrast agent can be accurately distinguished from unbound contrast agent.

Such gas filled bubbles can be prepared from a production fluid containing a halogenated solvent, a low concentration of shell forming biodegradable polymer, a second non-polar liquid with not too high a molecular weight which will allow for removal by lyophilization. Biodegradable polymers are chosen that are insoluble in the receiving liquid, but also insoluble in the production fluid if the halogenated solvent has disappeared by diffusion into the receiving liquid followed by evaporation. Upon lyophilization the second, non polar solvent is removed by sublimation leaving hollow particles

Typical biodegradable polymers that can be used in the invention are biopolymers, such as dextran and albumin or synthetic polymers such as poly(L-lactide acid) (PLA)and certain poly(meth)acrylates polycaprolacton, polyglycolicacid Of particular importance are so-called (block)copolymers that combine the properties of both polymer blocks (e.g. hydrophobic and hydrophilic blocks). Examples of random copolymers are poly(L-lactic-glycolic acid)(PLGA) and poly(d-lactic-1-lactic acid) Pd,lLA; Examples of diblock copolymers are poly(ethylene glycol)-poly(L-lactide) (PEG-PLLA), poly(ethylene glycol) -poly(N-isopropylacryl amide)(PEG-PNiPAAm)and poly(ethylene oxide)-poly(propylene glycol) (PEO-PPO). An example of a triblockcopolymer is poly(ethylene oxide)-poly(propylene glycol)-poly(ethyleneoxide) (PEO-PPO-PEO).

Good results are achieved when a polymer, such as an L-polylactide, with a fluorinated end group, such as C₆F₁₄ is employed in the production fluid. For the preparation of hollow capsules this is especially advantageous. If the inside of a capsule is hydrophobic, there will be no tendency to the condensation of water vapour on the inside wall of the capsules. Therefore, the capsules will not fill up with water but remain gas-filled for long periods of time, which is desirable for an ultrasound contrast agent. Incorporating fluor containing groups in the polymer increases the hydrophobicity of the inside capsule wall, and therefore inhibits condensation. In addition the incorporation of fluor containing groups gives a more efficient diffusion barrier for water and polar solutes

The micro-spheres that result from this production liquid have a very good impermeability for water The synthesis of such fluorinated polymer is known per se from the U.S. Pat. No. 6,329,470.

The elasticity of the shell can be tuned by varying the polymer properties, the important parameters or the gel transition temperature and the maximum elongation before breakage of the a film made from the material will occur.

Micro-spheres filled with a liquid such as a fluorinated liquid, such as perfluorobromo-octane are not only useful for ultrasound but also for functional magnetic resonance imaging (fMRI). The technique of fMRI is generally disclosed in the Proc. Intl. Soc. mag. Reson. Med. 9 (2001) 659-660. In particular on then basis of the nucleus ¹⁹F magnetic resonance spectroscopy measurements can be made of tissue oxygenations, pharmacokinetics of fluorinated cancer drugs as mentioned per se in the Proc. Intl. Soc. mag. Reson. Med. 9 (2001) 497 They can be prepared as described above, except that fluorine containing non-polar liquid is chosen and that this liquid is not removed during lyophilization.

Micro-spheres can also be filled with drugs; drugs can be dissolved in an oil, and micro-spheres with a liquid core will be formed, or gaseous drugs can be incorporated by exposing micro-spheres to the gas containing the gaseous drug after lyophilization. The drugs can be used for controlled release, for instance release by an ultrasound pulse to effectuate local delivery. This will be most efficient when targeted micro-spheres are used.

Drugs can also be incorporated in (otherwise) dense micro-spheres. Notable radio-active compounds, such as (activated/chelated) Holmium compounds for the treatment of liver malignancies are useful. For example Holmium finctions as a magnetic resonance contrast agent which induces T₁ as well as T₂ contrast. Further, Holmium can made radioactive by irradiating with neutrons. The radioactive isotopes of Holmium irradiate β-radiation (high-energy electronics) as well as γ-radiation. The β-radiation can be employed therapeutically to locally destroy tumours while the activity as magnetic resonance contrast agent enables monitoring of correct local application of the radioactive Holmium. Additionally the γ-emission can be detected by a gamma-camera to image the anatomy where the Holmium is applied. Micro-spheres with non-radioactive Holmium are first formed and subsequently by irradiating with neutrons the Holmium in converted into radioactive Holmium isotopes in the micro-spheres. The Holmium should not be released until it has lost its radioactivity. Particle should be big enough to get trapped in the capillary bed and no fine micro-spheres should get a chance to circulate in the blood. For this reason a well controlled synthesis is required.

The typical size of the micro-spheres depends on the specific application. Preferred sizes range from 1-100 μm. For example micro-spheres for US imaging as blood-pool agents have most preferred diameters between 1-10 μm. Most preferred diameters for Holmium encapsulated micro-spheres are within 15-40 μm.

These and other aspects of the invention are further elaborated with reference to the detailed examples and with reference to the accompanying drawing wherein

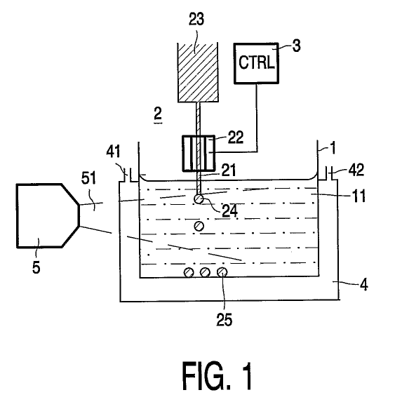

FIG. 1 shows a diagrammatic representation of a system for manufacturing micro-bubbles of the invention;

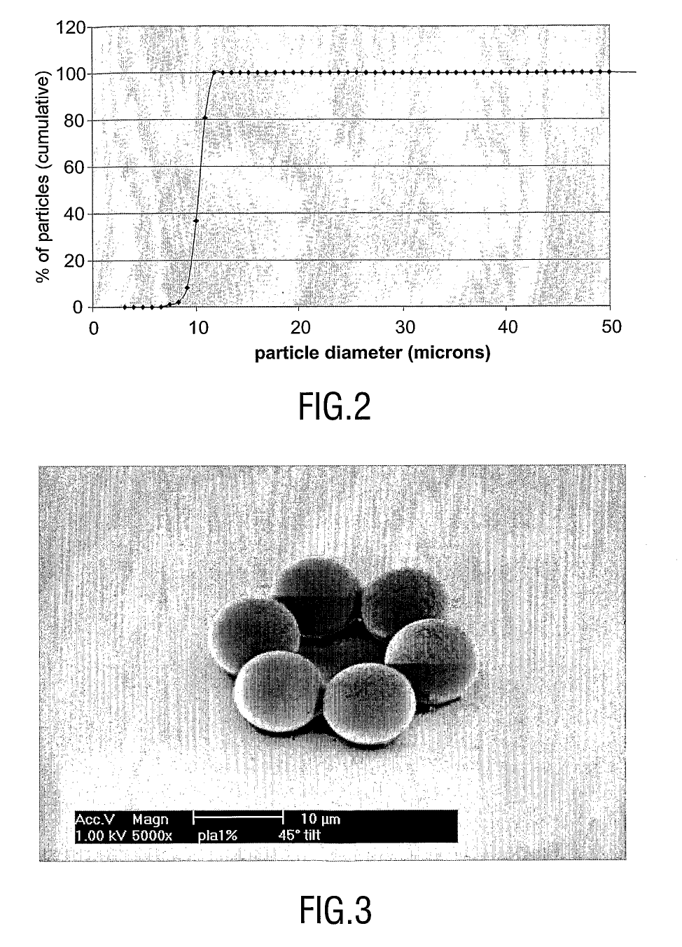

FIG. 2 shows the size distribution of inkjetted particle after washing with PVA, percentage of particle in 1 μm classes is given;

FIG. 3 shows a SEM picture of PLA particles obtained according to the procedure described in Example 1 below and

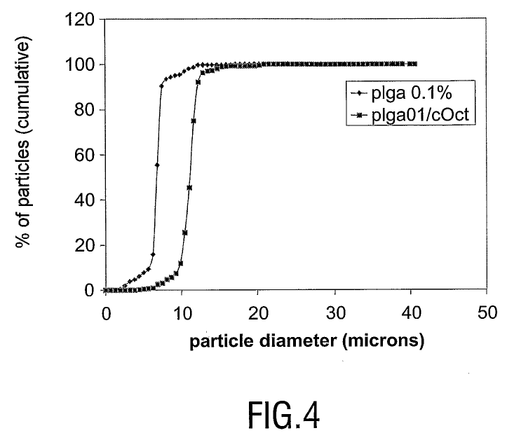

FIG. 4 shows size distributions from Examples 7 (0.1% plga) and 8 (0.1% plga, 0.3% cyclo-octane);

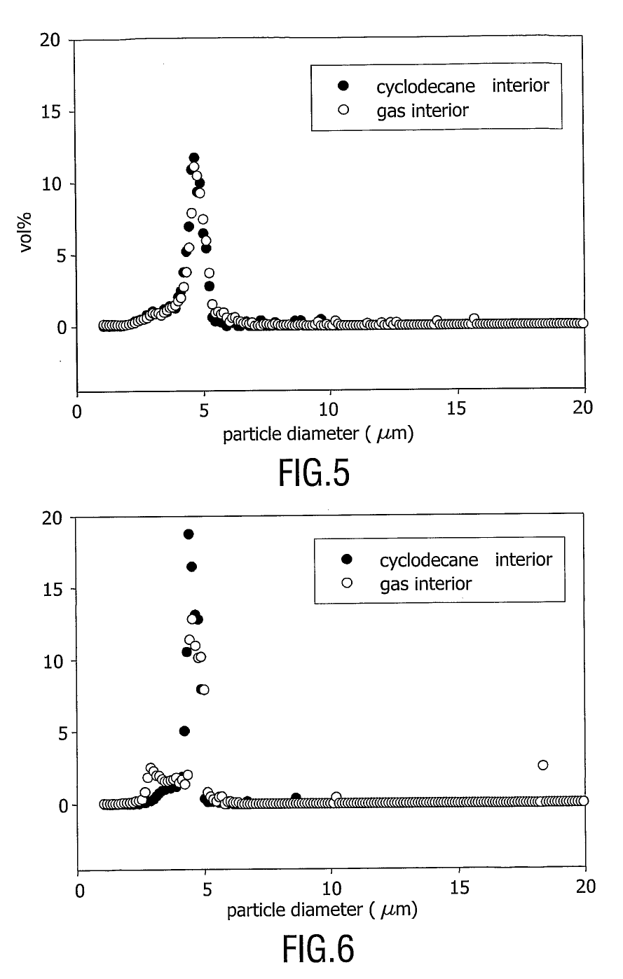

FIG. 5 shows an example of microspheres made of an L_polylactide having a model diameter of 4.7 μm;

FIG. 6 shows an example of microspheres made of an L_polylactide having a model diameter of 4.51 μm.

FIG. 1 shows the diagrammatic representation of a system for manufacturing micro-bubbles of the invention. The system for manufacturing micro-bubbles comprises the reservoir 1 which contains the receiving fluid 11. A jetting system 2 includes a nozzle 21 to eject of jet droplets of the production fluid 23 into the receiving fluid. The nozzle 21 is provided with a piezo-electrical system 22 that applies pressure pulses to the nozzle to produce the droplets 24 from which the micro-spheres 25 form that assemble in this example at the bottom of the reservoir 1. For example the nozzle 21 may be configured an ink-jetting head.

The jetting system 2 is also provided with a control unit 3 which applies electrical pulses to the piezo-electrical system 22. The control unit in this way controls the operation of the jetting system to produce the droplets of the production fluid.

Further, a cooling system 4 is provided, in this example in the form of a jacket 4 through which a cooling fluid, e.g. water, is passed from an inlet 41 to an outlet 42. The cooling system operates to cool the receiving liquid to below room temperature.

Additionally, the system for manufacturing micro-bubbles is provided with an ultraviolet radiation source 5, which emits a (pulsed) beam of ultraviolet radiation to the droplets of production fluid from the nozzle to cause photoinitiasation of polymerization in the droplets in order that micro-spheres are formed.

### EXAMPLES

### Example 1

### Preparation of 10 mm PLA Particles

A 1% PLA (poly-DL-lactide, Aldrich) solution in dichloroethane was inkjetted, starting immediately after immersion of the ink jet head into an aqueous 1% PVA (15/79) solution in a fluorescence cuvet. The initial drop diameter is about 50 μm as observed through the cuvet, which corresponds to a drop volume of 6.5*10-14 m3. After inkjetting for 20 minutes at 1,500 Hz, the procedure was stopped. The sediment was redispersed and transferred to a glass sample bottle and stirred for one hour to remove the dichloroethane. The particles were washed 3 times with filtered (200 nm), deionised water. A sample was taken for microscopic examination, revealing well dispersed spherical particles with a diameter of about 10 μm. The size distribution obtained from microscopic examination using a 20× objective and image pro plus software to analyze the mean diameter is given in FIG. 2. The sample was freeze dried for 48 hours and stored at −20° C. SEM pictures, taken after redispersion in filtered deionised water, drying and deposition of a 3 nm Pd/Pt layer, show a particle size of 10.2±0.3 μm which corresponds to a particle volume of 5.6*10-16 m3. As the densities of dichloroethane and PLA are approximately equal, the volume ratio between initial and final size demonstrates that PLA particles have been prepared with a low porosity. An SEM picture of the particles produced is given in FIG. 3.

### Example 2

### Preparation of 18 mm PLA Particles

A 3% PLA (poly-DL-lactide, Aldrich) solution in dichloroethane was inkjetted, starting immediately after immersion of the ink jet head into a aqueous 1% PVA solution in a fluorescence cuvet. After inkjetting for 20 minutes at 1,500 Hz, the procedure was stopped. The sediment was redispersed and transferred to a glass sample bottle and stirred for one hour to remove the dichloroethane. The particles were washed 3 times with filtered (200 nm), deionised water. A sample was taken for microscopic examination, revealing well dispersed monodisperse spherical particles with a diameter of about 18 μm. Freeze drying did not change the particle size. The volume ratio between initial droplet volume and final particle size is 20, which is expected for a 5% solution if completely dense polymer particles would have formed. This indicates that remaining porosity is present in these prepared particles made from a 3% solution.

### Example 3

### Preparation of PLGA Particles

A 3% PLGA (Poly-DL lacticde-co-glycolide (75:25), Aldrich) solution in dichloroethane was inkjetted, starting immediately after immersion of the ink jet head into a aqueous 1% PVA solution in a fluorescence cuvet. After inkjetting for 20 minutes at 1,500 Hz, the procedure was stopped. The sediment was redispersed and transferred to a glass sample bottle and stirred for one hour to remove the dichloroethane. The particles were washed 3 times with filtered (200 nm), deionised water. A sample was taken for microscopic examination, revealing well dispersed monodispersed spherical particles with a diameter of about 18 mm. Freeze drying did not change the particle size. The volume ratio between initial droplet volume and final particle size is 20, which is expected for a 5% solution if completely dense polymer particles would have formed. This indicates that remaining porosity is present in these prepared particles made from a 3% solution.

### Example 4

### Preparation of pla Particles Using Continuous Inkjet

A 1% solution of pla in dichloroethane was prepared and inkjetted into a 1% aqueous PVA 15/79 solution at a frequency of 14 kHz using a 50 μm nozzle. After evaporation of dichloroethane, washing and freeze-drying particles with an average diameter of 15.3 μm and a standard deviation of 2.7 μm were formed as quantified using image analysis of optical microscopy pictures.

### Example 5

### Preparation of pla Particles Loaded with Holmium-acetylacetonate

A 1% solution of pla, 0.02% of holmium-acetylacetonate in dichloroethane was inkjetted into a 1% aqueous PVA (15/79) solution at a frequency of 14 kHz using a 50 μm nozzle. The particles formed after evaporation of dichloroethane, washing and freeze-drying had an average diameter of 15.7 μm and a standard deviation of 2.6 μm as quantified using image analysis of optical microscopy pictures.

### Example 6

### Preparation of 12 mm plga Particles by Continuous Inkjet

A 1% solution of plga (75% lactic acid, 25% glycolic acid) in dichloroethane was prepared and inkjetted into a 1% PVA 15/79 solution at a frequency of 14 kHz using a 50 μm nozzle. The particles formed after evaporation of dichloroethane, washing and freeze-drying had an average diameter of 12.5 μm and a standard deviation of 2.3 μm as quantified using image analysis of optical microscopy pictures.

### Example 7

### Preparation of 7 mm plga Particles by Continuous Inkjet

A 0.1% solution of plga (75% lactic acid, 25% glycolic acid) in dichloroethane was prepared and inkjetted into a 1% PVA 15/79 solution at a frequency of 14 kHz using a 50 μm nozzle. The particles formed after evaporation of dichloroethane, washing and freeze-drying had an average diameter of 6.8 μm and a standard deviation of 1.3 μm, as quantified using image analysis of optical microscopy pictures. The size distribution is indicated in FIG. 4.

### Example 8

### Preparation of 11 Micron Polymer-shelled Capsules

A 0.1% solution of plga and 0.3% of cyclo-octane ) in dichloroethane was prepared and inkjetted into a 0.1% PVA 40/88 solution at a frequency of 14 kHz using a 50 μm nozzle. Dichloroethane was evaporated, the sample was washed with water previously saturated with cyclo-octane, and freeze-dried. Capsules with a diameter of 11.2 μm with a standard deviation of 1.8 μm were formed, as quantified using image analysis of optical microscopy pictures, the size distribution is indicated in FIG. 4. Capsules had a smooth surface contained one single cavity as deduced from SEM pictures.

### Example 9

### Preparation of Lipid-coated Capsules

A 0.1% plga, 0.3% cyclooctane, 0.005% asolectin in dichloroethane was inkjetted into an aqueous PVA 15/79 solution at 12 kHz using a 50 μm nozzle. The dichloroethane was evaporated, the sample was washed and freeze dried, smooth capsules with a diameter of 7.5 μm were observed using SEM exhibiting a single hollow core.

### Example 10

An L-polylactide having a C₆F₁₄ end group was dissolved at a concentration of 0.01% in dichloroethane in the presence of 0.01% cyclodecane. Using submerged inkjetting in 0.3% pva with a 50 μm nozzle at a frequency of 23,000 Hz droplets were formed with an initial diameter of about 85 μm. By repeated washing and stirring overnight the droplets shrank to form cyclodecane filled capsules with a modal diameter of 4.7 μm. The size distribution was measured on a Coulter Counter and is given in FIG. 5. The sample was lyophilised to remove the core of cyclodecane, the size distribution after removal and redispersion is unchanged as shown in FIG. 5. Microscopy on redispersed samples showed gas filled capsules. Upon exposure to ultrasound the escape of gas could be detected

### Example 11

An L-polylactide having a C₆F₁₄ end group was dissolved at a concentration of 0.005% in dichloroethane in the presence of 0.01% cyclodecane. Using submerged inkjetting in 0.3% pva with a 50 μm nozzle at a frequency of 23,000 Hz droplets were formed with an initial diameter of about 85 μm. By repeated washing and stirring overnight the droplets shrank to form cyclodecane filled capsules with a modal diameter of 4.5 μm. The size distribution was measured on a Coulter Counter and is given in FIG. 6. The sample was lyophilised to remove the core of cyclodecane, the size distribution after removal and redispersion is hardly changed as shown in FIG. 6. Microscopy on redispersed samples showed gas filled capsules. Upon exposure to ultrasound the escape of gas could be detected.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. A System for manufacturing micro-spheres of a production fluid containing a constituting material, the system comprising:
a reservoir for holding a receiving fluid
a jetting module having at least one nozzle for jetting the production fluid into the receiving fluid,
Wherein the production fluid contains a concentration of the constituting material in the range between 0.01% and 5%.

16. The system of claim 15, wherein the system includes a control system to control the jetting at a jetting rate in the range of 100 kHz⁻¹ to 0.1 kHz.₋₁

17. The system of claim 16, wherein the control system is arranged to operate the jetting in a pulsed fashion so as to apply block form excitation pulses to the jetting module.

18. The system of claim 15, wherein the jetting system has several nozzles and the control system is arranged to adjust droplet-sizes for individual nozzles.

19. The system of claim 15, wherein the reservoir has a temperature control system.

20. The system of claim 15, wherein the system includes an irradiation module to irradiate the micro-spheres with electro-magnetic radiation of which the wavelength is in the range of 200-800 nm.

21. The system of claim 15, wherein a flight path of the micro-spheres extends from the nozzle into the receiving fluid over a distance.

22. The system of claim 15, wherein the receiving liquid and/or the production fluid contains a stabilizer from the group of lipids, surfactants, polymers or block copolymers.

23. An ultra-sound contrast agent comprising essentially monodisperse micro-spheres.

24. The ultrasound contrast agent of claim 23, wherein the ultrasound contrast agent is targeted to a specific location in the vasculature.

25. The ultrasound contrast agent of claim 23, wherein the ultrasound contrast agent is modified with peptides.

26. An MR-contrast agent comprising essentially monodisperse micro-spheres with an¹⁹F compound.

27. An encapsulated drug comprising essentially monodisperse micro-spheres loaded with a pharmaceutical active compound.

28. An encapsulated therapeutic compound comprising essentially monodisperse micro-spheres loaded with a radioactive compound or a compound having radioactive isotopes.

29. The ultrasound contrast agent of claim 23, wherein the ultrasound contrast agent targets at least one of thrombosis, vulnerable plaque or angiogenesis.

30. The ultrasound contrast agent of claim 23, wherein the ultrasound contrast agent is modified with at least one of antibodies or antibody fragments.

31. The drug of claim 27, wherein the pharmaceutical active compound is active against disease addressable from vasculature.

32. The therapeutic compound of claim 28, wherein the radioactive isotopes include Holmium.
